(19) United States
(12) Patent Application Publication
(10) Pub. No.: US 2026/0198969 A1
(43) Pub. Date: Jul. 16, 2026

(54) SURGICAL BONE FRAME ASSEMBLY

(71) Applicant: Automated Intelligent Robotics LLC, Sarasota, FL (US)

(72) Inventors: Thomas Medrick Sweeney II, Myakka City, FL (US); Michael Todd Boyer, Venice, FL (US); John Douglas Kuczynski, Sarasota, FL (US); Thomas Medrick Sweeney III, San Diego, CA (US); Daniel Davenport, Collegeville, PA (US); Vincent E. Mandes, Warrington, PA (US)

(73) Assignee: Automated Intelligent Robotics LLC, Sarasota, FL (US)

(21) Appl. No.: 19/375,373

(22) Filed: Oct. 31, 2025

Related U.S. Application Data
(60) Provisional application No. 63/833,179, filed on Nov. 1, 2024.
(60) Provisional application No. 63/871,709, filed on Aug. 28, 2025.

(51) Int. Cl. A61B 17/64; A61B 90/00

(52) U.S. Cl. CPC A61B 17/645; A61B 2090/3916

(57) ABSTRACT

An exemplary surgical bone frame assembly includes a frame, a frame fastener and a bone support assembly. The frame may be an external frame having a plurality of frame segments with adjustable connectors for operatively and pivotably connecting to each other. The frame fastener secures the frame to a patient. The bone support assembly is connectable to the frame for fastening the frame to a bone of a patient, and may include a bone support frame for rigidly connecting to the frame, and a bone fastener. The bone fastener extends from the bone support frame and securely attaches to the bone of the patient. The surgical bone frame assembly provides structural support and positioning control for anatomical regions during surgical procedures by creating rigid connections between multiple anatomical landmarks.

### CROSS REFERENCE TO RELATED APPLICATIONS

The present application claims the benefit of U.S. Provisional Patent Application No. 63/833,179 entitled “AUTOMATED DYNAMIC PATIENT POSITIONER FOR USE IN ROBOTIC SURGERY,” and U.S. Provisional Patent Application No. 63/871,709 entitled “SEGMENTAL REGISTRATION FOR BONE TRACKING,” the entire disclosures of which are hereby incorporated by reference in their entirety for all purposes.

### BACKGROUND

The present disclosure relates to patient positioning systems for surgical procedures, and more particularly to surgical frame assemblies that stabilize bone structures and maintain patient and/or bone positioning during surgery.

Robotic surgery has become increasingly prevalent in modern medical practice, offering enhanced precision, reduced invasiveness, and improved patient outcomes. However, a significant challenge in robotic surgery is maintaining consistent patient positioning throughout the procedure. Patients may move due to breathing, muscle relaxation under anesthesia, or external forces applied during surgery. Even small movements can compromise the accuracy of robotic surgical systems, potentially leading to suboptimal outcomes or the need to re-register the patient's position.

Current patient positioning systems are primarily static, consisting of articulating arms, pads, straps, and sometimes motorized actuators. While some dynamic positioning systems exist, these systems still allow undesired patient movement due to both internal forces (e.g., breathing) and external forces (e.g., instrumentation). As such, there still exists a need for a patient positioning system that can work in conjunction with robotic surgical systems to continuously monitor and maintain proper patient positioning throughout surgical procedures.

### BRIEF SUMMARY

The following presents a simplified summary in order to provide a basic understanding of some aspects of one or more embodiments or examples of the present teachings. This summary is not an extensive overview, nor is it intended to identify key or critical elements of the present teachings, nor to delineate the scope of the disclosure. Rather, its primary purpose is merely to present one or more concepts in simplified form as a prelude to the detailed description presented later. Additional goals and advantages will become more evident in the description of the figures, the detailed description of the disclosure, and the claims.

The foregoing and/or other aspects and utilities embodied in the present disclosure may be achieved by providing a surgical bone frame assembly useable for adjustably maintaining patient positioning. The system includes a frame, a frame fastener and a bone support assembly. The frame includes a plurality of frame segments having adjustable joints for operatively and pivotably connecting to each other. The frame fastener secures the frame to a patient. The bone support assembly is connectable to the frame for fastening to a bone of a patient. In examples, the bone support assembly includes a bone support frame for rigidly connecting to the frame, and a bone fastener extending from the bone support frame for securely attaching to the bone of the patient.

According to aspects illustrated herein, the bone fastener may include a connector that includes at least one off a pin, a screw and a clamp for attachment to the bone. The clamp may include a threaded screw, a C-clamp, a jaw clamp and/or a pair of opposing pivotable clamp arms, with each arm of the pair having a bone support frame with a serrated inner surface for operatively gripping the bone.

Another aspect of the exemplary embodiments is that the assembly may include fiducial markers on the frame and its components and a navigation system having navigation sensors for detecting the position of the fiducial markers.

According to yet other aspects of the exemplary embodiments, the plurality of frame segments may include a first plurality of frame members extending between a pair of the plurality of frame segments. In still other aspects, the bone support frame of the bone support assembly includes a rod and an adjustable connector articulatively and fixedly connecting the rod to the bone fastener. In yet still other aspects, the bone support frame of the bone support assembly includes a lattice structure of a plurality of frame segments connected via adjustable connectors therebetween, the adjustable connectors articulatively and fixedly connecting the frame segments to the bone fastener.

According to other aspects described herein, a surgical bone frame assembly for surgical procedures includes a bone support assembly for securing an orientation of unsettled bone segments of a patient. The bone support assembly may include a bone support frame, a plurality of bone fasteners extending from the bone support frame for securely attaching to the bone segments of the patient, and adjustable connectors articulatively and fixedly connecting the bone support frame to the plurality of bone fasteners. The bone support frame may include a plurality of rigid frame segments, with the adjustable connectors being operational to allow positioning of the rigid frame segments relative to the plurality of bone fasteners and to hold the rigid frame segments in a fixed configuration with the plurality of bone fasteners.

Exemplary embodiments are described herein. It is envisioned, however, that any system that incorporates features of apparatus and systems described herein are encompassed by the scope and spirit of the exemplary embodiments. Therefore, the foregoing general description of the illustrative embodiments and the following detailed description thereof are merely exemplary aspects of the teachings of this disclosure and are not restrictive.

### BRIEF DESCRIPTION OF THE DRAWINGS

The foregoing summary, as well as the following detailed description of the exemplary embodiments of the subject disclosure, will be better understood when read in conjunction with the appended drawings. For the purpose of illustrating the subject disclosure, there are shown in the drawings exemplary embodiments. It should be understood, however, that the exemplary embodiments are not limited to the precise arrangements and instrumentalities shown. In the drawings:

FIG. 1 is a perspective view of a surgical bone frame assembly in accordance with an exemplary embodiment of the subject disclosure;

FIG. 2 is a perspective view of the surgical bone frame assembly of FIG. 1;

FIG. 3 is another perspective view of the surgical bone frame assembly of FIG. 1;

FIG. 4 is an enlarged exploded perspective view of frame segments of a frame shown in FIG. 1;

FIG. 5 is an enlarged perspective view of another frame segment of the frame shown in FIG. 1;

FIG. 6 is an enlarged top perspective view of a frame fastener of the frame shown in FIG. 1;

FIG. 7 is an exploded view of a bone support assembly of the surgical bone frame assembly shown in FIG. 2;

FIG. 8 is an enlarged perspective view of a bone support frame of the bone support assembly of FIG. 2;

FIG. 9 is an exploded partial view of the surgical bone support assembly of FIG. 8;

FIG. 10 is an enlarged cross-sectional view of a tulip connector of the surgical bone support assembly shown in FIG. 8;

FIG. 11 is an enlarged perspective view of a bone fastener of the bone support assembly shown in FIG. 8;

FIG. 12 is an exploded view of the bone fastener shown in FIG. 11;

FIG. 13 is a perspective view of an exemplary bone fastener in accordance with another exemplary embodiment of the subject disclosure;

FIG. 14 is a perspective view of another exemplary bone fastener in accordance with another exemplary embodiment of the subject disclosure;

FIG. 15 is a perspective view of yet another exemplary bone fastener in accordance with another exemplary embodiment of the subject disclosure;

FIG. 16 is a perspective view of still another exemplary bone fastener in accordance with another exemplary embodiment of the subject disclosure; and

FIG. 17 is a perspective view of additional exemplary bone fasteners in accordance with examples of the embodiments of the subject disclosure.

### DETAILED DESCRIPTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale.

We initially point out that description of well-known starting materials, processing techniques, components, equipment and other well-known details may merely be summarized or are omitted so as not to unnecessarily obscure the details of the present disclosure. Thus, where details are otherwise well known, we leave it to the application of the present disclosure to suggest or dictate choices relating to those details. For example, the components of the surgical bone frame assembly may be molded, machined or 3D printed as well understood by a skilled artisan. The drawings depict various examples related to exemplary embodiments of illustrative methods, apparatuses, and systems for surgical bone frame assemblies useable with monitoring and positioning systems.

Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “controller” or “control system” is used herein generally to describe various apparatus such as a computing device relating to the operation of one or more device that directs or regulates a process or machine. A controller can be implemented in numerous ways (e.g., such as with dedicated hardware) to perform various functions discussed herein. A “processor” is one example of a controller which employs one or more microprocessors that may be programmed using software (e.g., microcode) to perform various functions discussed herein. A controller may be implemented with or without employing a processor, and also may be implemented as a combination of dedicated hardware to perform some functions and a processor (e.g., one or more programmed microprocessors and associated circuitry) to perform other functions. Examples of controller components that may be employed in various embodiments of the present disclosure include, but are not limited to, conventional microprocessors, application specific integrated circuits (ASICs), and field-programmable gate arrays (FPGAs).

Embodiments as disclosed herein may also include computer-readable media for carrying or having computer-executable instructions or data structures stored thereon. Such computer readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures.

When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, objects, components, and data structures, and the like that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described therein.

Although embodiments of the invention are not limited in this regard, discussions utilizing terms such as, for example, “processing,” “tracking,” “computing,” “calculating,” “determining,” “using,” “establishing”, “analyzing”, “checking”, or the like, may refer to operation(s) and/or process(es) of a controller, computer, computing platform, computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Furthermore, the described features, advantages and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

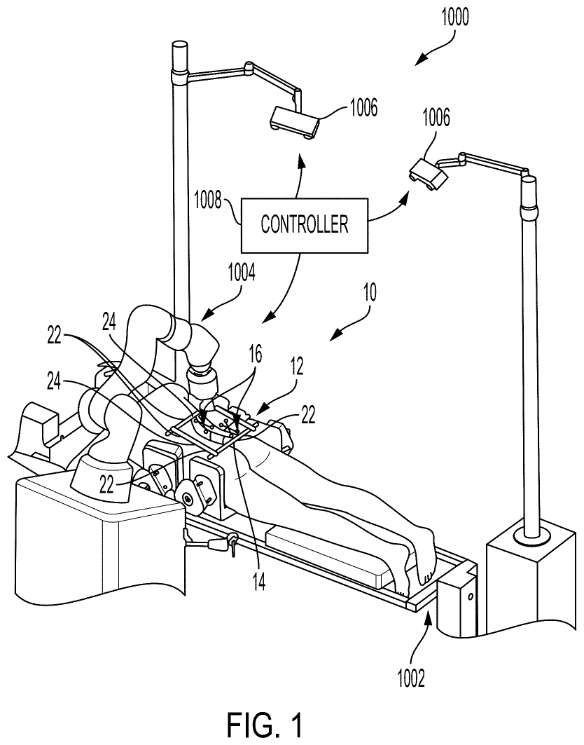

FIG. 1 illustrates an exemplary surgical bone frame assembly 10 that may be used with various patient positioning systems, such as patient positioning system 1000 for surgical procedures. The patient positioning system 1000 may include a base 1002 for supporting a patient, a robotic end effector 1004, a navigation system 1006, and a controller 1008. The patient, shown lying prone may be in any position suitable for a given surgical procedure.

The surgical bone frame assembly 10 includes a frame 12, a frame fastener 14, and a bone support assembly 16. The bone support assembly 16 includes a bone support frame 18 for rigidly connecting to the frame and a bone fastener 20 extending from the bone support frame for securely attaching to the bone of a patient. The surgical bone frame assembly 10 provides structural support and positioning control for various anatomical regions during surgical procedures by creating rigid scaffolding between various bones of the patient.

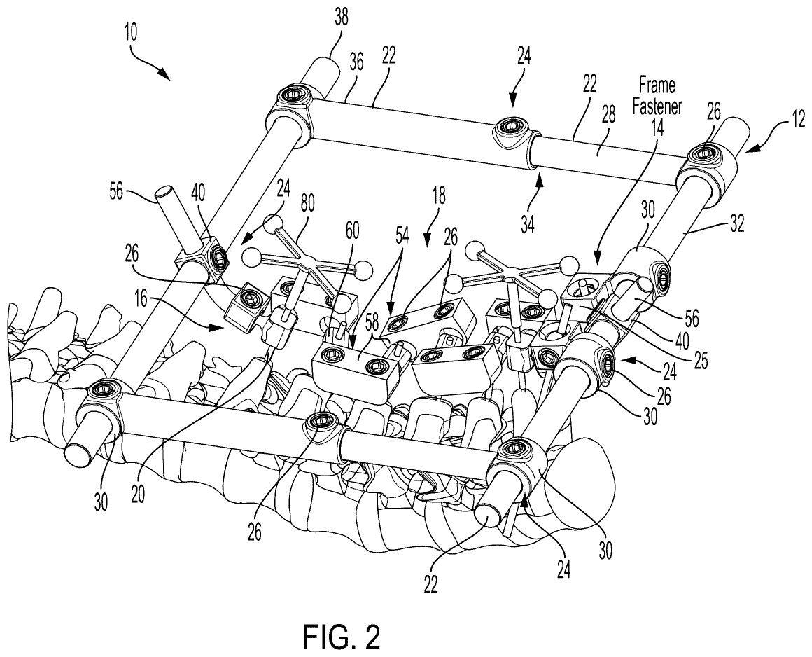

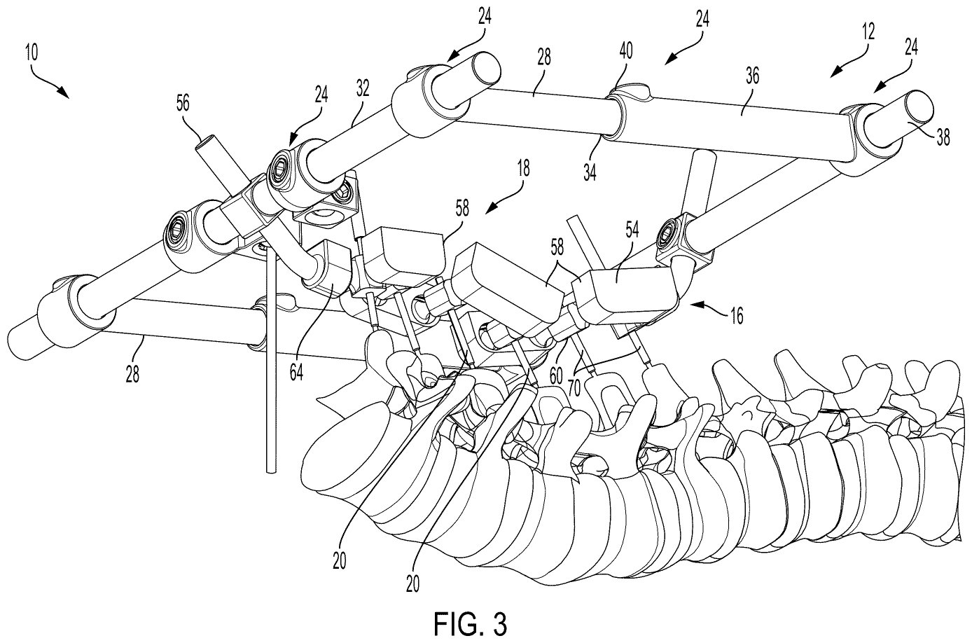

FIGS. 2 and 3 are enlarged views of the surgical bone frame assembly 10. While not being limited to a particular theory, the surgical bone frame assembly 10 provides stabilization and tracking capabilities for maintaining patient anatomical positioning. The frame assembly may be configured to mount to various anatomical structures of a bone (e.g., iliac, spinous processes, etc.) and provide direct skeletal fixation positioning stability.

The surgical bone frame assembly 10 includes the frame 12 having a plurality of frame segments 22 and adjustable joints or adjustable connectors 24 for operatively and pivotably connecting each of the individual frame segments for forming a rigidly connected and stable frame. The frame 12 may be an exterior frame positioned outside a patient. While the frame 12 is shown as generally square or rectangular, it is understood that the frame is not limited to any particular shape or size, and may be oriented to accommodate variations in patient anatomy and positioning requirements while maintaining structural integrity throughout surgical procedures.

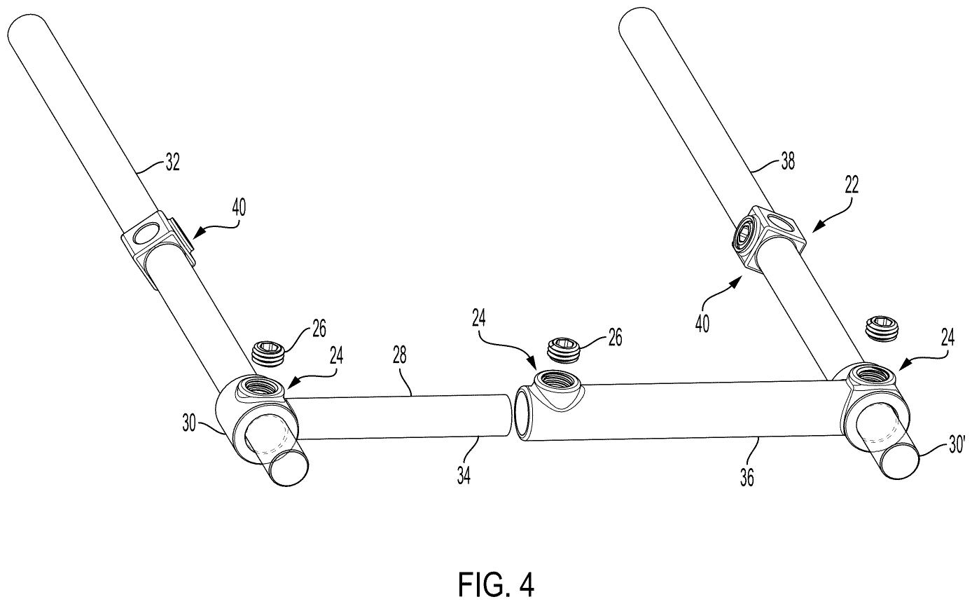

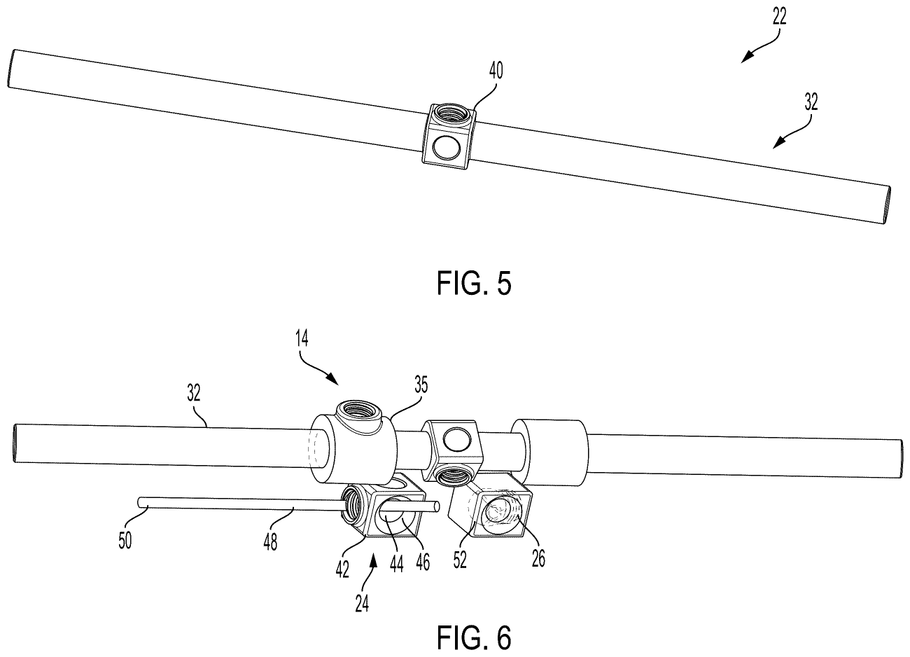

FIGS. 4 and 5 depict exemplary frame segments 22, with FIG. 4 showing frame segments in exploded and phantom views. The frame segments 22 may include elongated shafts or rods that are strong yet lightweight for coupling to a patient and placement external to a patient anatomical area of interest. For example, the frame segments may have a tensile strength of about 10,000 to 800,000 psi, a specific strength of about 60 to 2500 MPa·cm³/g, a bending modulus of at least 50 GPa, or about 65 to 200 GPa, and an overall weight of each frame segment of about 0.02 to 15 kg, or about 0.1 to 8 kg, or about 0.3, 0.5, 1, 2, 3, 4, 5, 6 or 7 kg. Additionally, each frame segment may have a length of about 1-20 inches, or 2-15 inches, or about 3, 4, 5, 6, 7, 8, 9, 10, 11, 12, 13 or 14 inches. The frame segments may be comprised of a metal (e.g., aluminum, titanium, stainless steel), a composite, a ceramic, a rigid polymer or any other material suitable for the intended use.

As shown in FIG. 4, the frame segments 22 include a first rod 28 having a solid core that terminates at a first end having a sleeve coupling connector 30 with an aperture for receiving and holding a distal transversely oriented frame segment 32. The sleeve coupling connector 30 also includes a threaded bore wall for receiving a locking actuator 26 (e.g. a set screw) that can be loosened to allow the transversely oriented frame segment 32 to move relative to the first rod 28, and tightened to abut and lock the frame segment 32 to the first rod. As such, the sleeve coupling connector 30 is operable to release and fixedly secure the first rod to the frame segment 32 in response to mechanical loosening or tightening inputs. The sleeve coupling connector 30, and locking actuator 26 collectively form an adjustable connector, or in other words, the adjustable connector comprises a sleeve coupling connector and locking actuator. The first rod 28 has a second end 34 that may be inserted into and coupled to a hollow or annular frame segment 36 having an axial bore that receives the second end.

The annular frame segment 36 includes a threaded bore wall for housing another locking actuator 26 that can be loosened to allow the first rod 28 freedom to move relative to the annular frame segment, and tightened to abut and lock the first rod to the annular frame segment. In other words, the locking actuator 26 is operable to release and fixedly secure the annular frame segment to the first rod in response to mechanical loosening or tightening inputs. The annular frame segment 36 further includes a sleeve coupling connector 30′ with an aperture 3

holding a proximal transversely oriented frame segment 38. The sleeve coupling connector 30′ also includes a threaded bore wall for receiving a locking actuator 26 operable to release and fixedly secure the annular frame segment to the proximal transversely oriented frame segment 38.

The sleeve coupling connectors and locking actuators 26 are exemplary adjustable connectors 24 that have interconnected aspects that may change the shape, size and/or stiffness of the frame in response to mechanical loosening or tightening of the connectors. While not being limited to a particular configuration, the locking actuators 26 can be any suitable fastener such as screws, set screws, clamps, and threaded caps, that fixedly engaged in an aperture of a connector or fitting either e.g., via threaded engagement, press-fit, or other locking means. After any two frame parts are connected by the adjustable connector 24, the adjustable connector may be tightened via the locking actuator to keep the connected frame segments fixed in orientation. Of course, other locking actuators (e.g., clamping couplings, taper lock bushings) that apply clamping force to an adjustable connector to prevent further movement of the connected frame members are available within the scope of the examples.

The distal transversely oriented frame segment 32 connected to the first rod 28 is shown in FIG. 5. The frame segment 32 is an elongated frame segment rod that can be of solid core configuration or annular, and includes a connector 40 having a bore extending through opposite ends of the connector for receiving the bone support assembly as will be discussed in greater detail below. The connector 40 can be disposed substantially centrally of the frame segment 32 and also include a threaded bore wall for receiving a locking actuator 26 operable to release and fixedly secure the frame segment 32 to the bone support assembly 16.

The proximal transversely oriented frame segment 38 connected to the annular frame segment 36 can be generally identical in structure to the distal transversely oriented frame segment 32.

The frame fastener 14 is configured as best shown in FIGS. 2, 3 and 6. The frame fastener operatively secures the frame 12 to the patient. The frame fastener 14 includes adjustable connectors 25. In accordance with an exemplary embodiment, the adjustable connector 25 includes a sleeve connector 35 attached to a ball and socket connector 42. The sleeve connector 35 encircles e.g., the distal frame segment 32, and includes a threaded bore wall for receiving and housing a threaded locking actuator 26 operable to release and fixedly secure the frame fastener 14 to the frame 12 in response to mechanical loosening or tightening inputs. The ball and socket connector 42 is an adjustable connector that includes a ball 44 within a connector socket 46 and a threaded bore wall for housing a locking actuator 26 operable to release and fixedly secure the ball to the socket housing in response to mechanical loosening or tightening inputs.

The ball 44 is a spherical clamp having a central bore to hold an elongated pin 48 and slots 52 to allow the ball to open or close and securely grip the pin. The elongated pin 48 is attached to the ball 44 and extends from the ball to a distal end 50 configured to attach securely to a bone (e.g., iliac, large bone, femur, etc.) and fix the frame fastener 14 to the patient. The distal end 50 may include a sharp or threaded screw tip for secure attachment to the patient bone, for example, by screwing into the bone. In some examples the distal end may include a clamp for clamping onto a bone.

Referring to FIGS. 2, 3 and 7, the bone support assembly 16 connects to the frame 12 for fastening one or more bones of the patient to the frame. More particularly, the bone support assembly connects to the bones of the patient to be immobilized or stabilized in position that is the subject of a surgery. The frame 12 may be secured generally around a perimeter of the bone support assembly 16, although, in examples, the bone support assembly may extend beyond the frame. In examples, the bone support assembly 16 attaches to a plurality of bones or bone segments (e.g., spinous process, fractured bone segments, adjacent unsettled bones) and secures the bones in a fixed orientation that may be further secured via rigid attachment to the frame. The bone support assembly 16 includes a bone support frame 18 for rigidly connecting to the frame, and one or more bone fasteners 20 extending from the bone support frame for securely attaching bones of the patient to the frame.

The bone support frame 18 may include a plurality of support frame members 54 that extend between a pair of the frame segments 22 and attach to the frame 12. The support frame members 54 are joined and form a lattice or grouping of interconnecting mechanical linkages (e.g., rods, shafts, pins, beams, joints, wire, clamps, connectors, housings) that can change the shape, size, and/or stiffness of the bone support assembly 16 in response to mechanical loosening or tightening inputs. FIGS. 2, 3 and 7 show exemplary support frame members 54 including elongated frame support rods 56, support frame links 58, and rod links 60 having ball connectors 62.

The elongated frame support rods 56 are shown at opposite ends of the bone frame assembly 16. Each frame support rod 56 includes an adjustable connector, such as a housing 64 with a connector socket 46 that receives a ball connector 62 of an adjacent rod link 60 and threaded bore walls 66. The threaded bore walls 66 extend to the connector socket 46, with the threaded bore walls each housing or configured to house a threaded locking actuator 26 operable to release and fixedly secure the ball connector 62 to the elongated frame support rod in response to mechanical loosening or tightening inputs. The elongated frame support rod 56 has a proximal end shaped to connect to the frame 12. For example, the elongated rod 56 can penetrate through the bore of the frame segment connector 40 and rigidly attach to the frame via locking actuator 26.

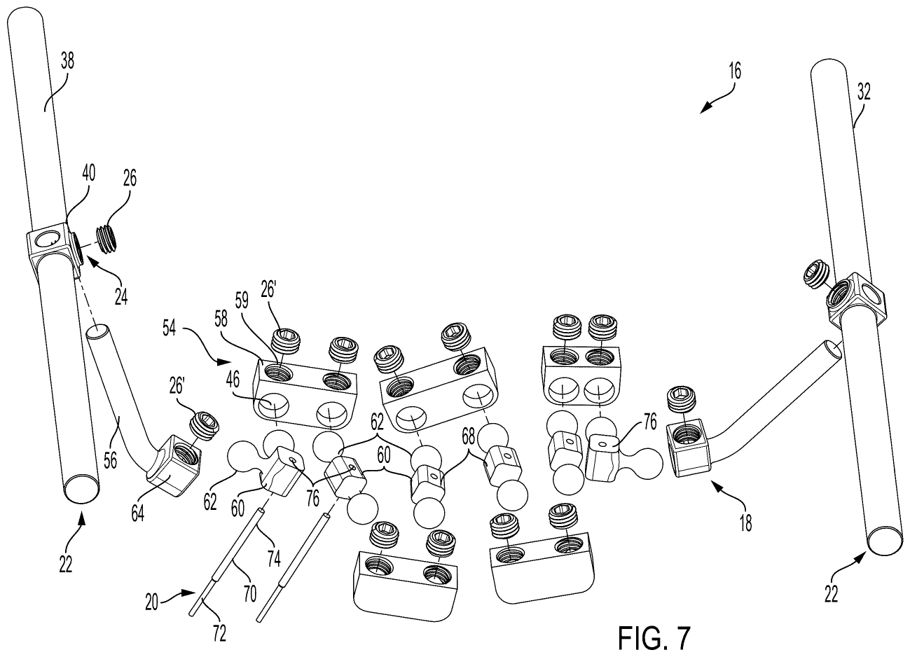

While not being limited to a particular configuration the support frame links 58 include housings 59, for example, rectangular prism shaped housings with one or more or a plurality of connector sockets 46. The support frame links 58 connect to rod links 60 that extend laterally between offsetting support frame links e.g., in a serpentine orientation. As such, the support frame links 58 and rod links 60 may include crossbars, shafts, rods, stems and other links configured for connection to form the lattice linkage. Each socket 46 is configured to receive a respective ball connector 62 of an adjacent rod link 60. As can best be seen in FIG. 7, each support frame link 58 includes two connector sockets 46 and two threaded bore walls 66 for housing a locking actuator 26′ operable to release and fixedly secure the ball connector 62 within a socket 46 in response to mechanical loosening or tightening inputs. The support frame links 58 may be spaced apart and offset extending longitudinally along an anatomical region such as along the length of a spine or plurality of adjacent bones (FIGS. 2 and 3).

Alternatively expressed, the bone support frame includes adjustable connectors 24 formed by the sockets, ball connectors and locking actuators of the support frame links 58, rod links 60 and locking actuators 26′ (e.g., screws, set screws, clamps, threaded caps, cam-actuated) that allow polyaxial movement when unlocked, and rigid fixation when locked.

Referring to FIG. 7, the rod link 60 includes a prism shaped body 68 with narrowed sectioned stems extending from the body section to bulbous or spherical shaped ball connectors 62. In some examples the ball connectors are at opposite ends of the rod link 60. In other examples, the ball connectors extend from the body section 68 at angles other than 180° (e.g., 90 degrees, perpendicular, acute, or obtuse relative to a longitudinal length of an adjacent ball connector).

While not being limited to a particular bone support frame configuration, in FIGS. 2, 3 and 7 the support frame links 58 include female apertures (e.g., connector sockets), and the rod links 60 include male connectors (e.g., bulbous connectors) configured to fit loosely in the female apertures to allow articulation therebetween. The support frame links 58 also include threaded bore walls in fluid communication with the female apertures. In alternative examples, the rod links may include female connectors that accept mating male connectors of support frame links and also include threaded bore walls for rigid attachment to the male connectors via locking actuators.

The bone support assembly 16 also includes one or more bone fasteners 20 that extend from the bone support frame 18 for anchoring or securely attaching to one or more bones of the patient. While not being limited to a particular theory, the bone fastener 20 may be an elongated member having a distal end that attaches to a bone or bone segments of the patient, and may hold the bone in a fixed position as desired during instrumentation. For example, in FIGS. 2 and 7, bone fasteners 20 is configured as pins 70 having a distal end with a piercing tip 72 that may penetrate a bone and remain in the bone via frictional engagement. The piercing tip 72 may be threaded to help secure the pin to the bone. The bone fastener 20 is not limited to this configuration, as the distal end may include other structure operable to securely hold a bone, such as a clamp, a wire or other fastener.

The bone fastener 20 includes a coupling body portion 74 operable to connect to the bone support frame 18. As can be seen in FIGS. 2, 3 and 7, the pin 70 has a longitudinal midsection 74 that attaches to a respective support frame member 54. In particular, the longitudinal midsection extends through or connects to the body section 68 of a rod link 60 via aperture 76. The aperture 76 can be configured to have a diameter about or slightly less than a diameter of the pin 70 longitudinal midsection 74. The aperture 76 is merely one example of a connector operable to connect the bone fastener 20 to the bone support frame 18. For example, adjustable connectors that loosen to allow movement between the bone fastener 20 and support frame members 54 and tighten for a rigid fixation therebetween are considered within the scope of the examples of the subject disclosure, as readily understood by a skilled artisan.

The frame segments 22, adjustable connectors 24, frame fastener 14, support frame members 54, and bone fasteners 20 may thus be adjustably connected and change shape, orientation, size and stiffness via interconnecting mechanical linkages in response to mechanical loosening or tightening inputs between adjustable and locked configurations. In an unlocked or loose configuration, the interconnected elements may be adjusted to match the patient's anatomy and body part geometry. Then, once a desired positioning of the patient's anatomy is achieved, locking actuators 26 may engage the adjustable connectors and secure the connections and create a rigid framework to retain the patient's anatomy in a predetermined or desired fixed position.

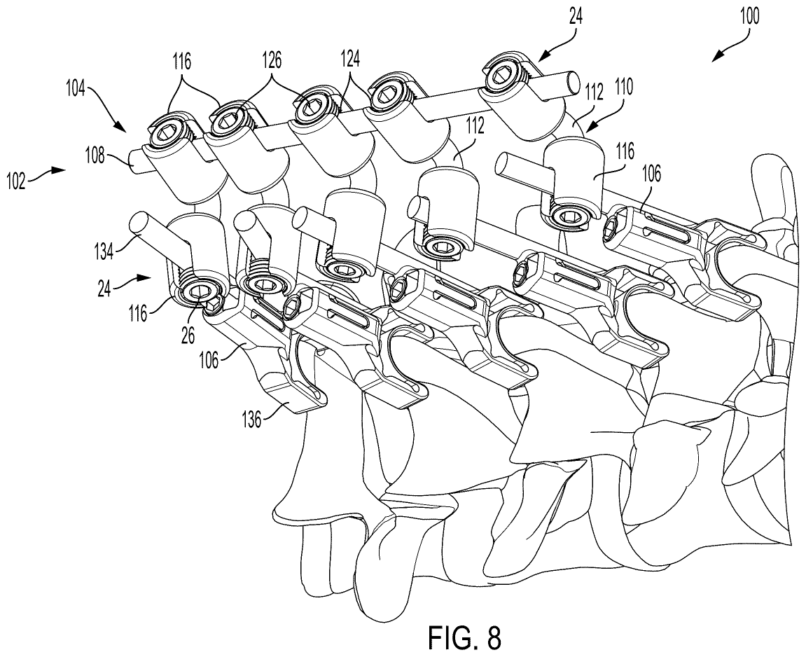

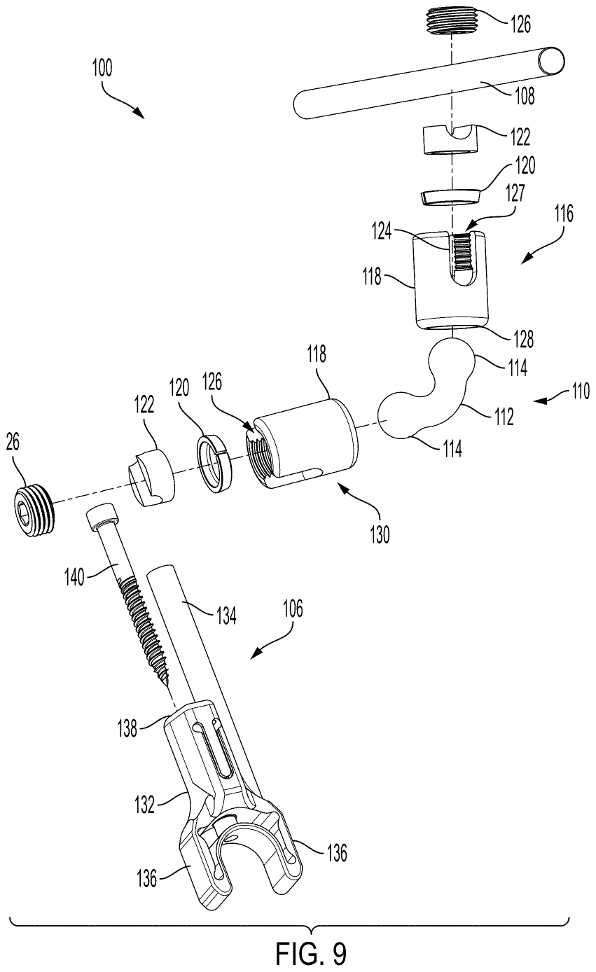

FIGS. 8 and 9 depict another exemplary embodiment of a surgical bone frame assembly 100 that provides stabilization and tracking capabilities for securing, adjusting and maintaining patient anatomical positioning. In the example illustrated, the surgical bone frame assembly 100 is attached to a patient's spinous processes and provides direct skeletal fixation positioning stability. While the frame assembly is shown extending along a portion of the spine, it is understood that the frame assembly may extend to any desired length of the spine or other bones. The frame assembly 100 includes a bone support assembly 102 for securing an orientation of unsettled bones of a patient. The bone support assembly 102 includes a bone support frame 104, bone fasteners 106 extending from the bone support frame for securely attaching to the patient's bone, and adjustable connectors 24, 25 that connect the bone support frame to the bone fasteners. The bone support assembly 102 may be attached to a frame and frame fastener similar to those of frame 12 and frame fastener 14 of FIG. 2.

The bone support frame 104 may include one or more support frame members that may extend the length of the surgical bone frame assembly 100 and may attach to the frame and/or frame fastener. The bone support frame includes a frame rod 108 having a longitudinal extent, e.g., for extending longitudinally along a spine. It is understood that the frame rod 108 may be a single unitary rod or one or more rods attached together and may extend along the spine or other anatomical bones of interest of a patient. The support frame members, including the frame rod 108, are strong yet lightweight for coupling to a patient. Each support frame member may have a length of about 0.1-20 inches, or 0.2-15 inches, or about 0.5, 1, 2, 3, 4, 5, 6, 7, 8, 9, 10, 11, 12, 13 or 14 inches. The frame rod may be comprised of a metal (e.g., aluminum, titanium, stainless steel), a composite, a ceramic, a rigid polymer or any other material suitable for the intended use.

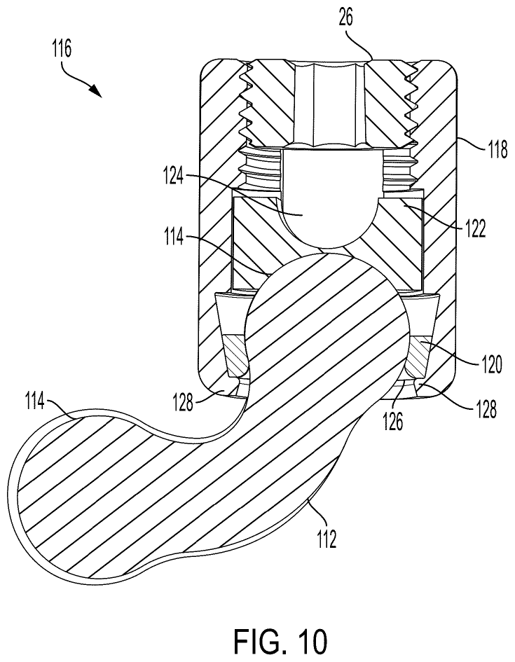

The frame rod 108 connects to extended range couplers 110 that may extend generally laterally from the frame rod for adjustable connection with bone fasteners 106, as will be discussed in greater detail below. The extended range couplers 110 include a generally cylindrical bent shaft 112 having a bend e.g. of about 30-150 degrees, including 40, 50, 60, 70, 80, 90, 100, 110, 120, 130, 140 degrees, and adjustable connectors 116 (e.g., tulip connectors). The shaft 112 is bent to allow articulation and spacing from the frame rod. As can be seen in FIGS. 9 and 10, the bent shaft 112 extends from its bend to enlarged bulbous or spherical ends 114 operable as coupling bearing surfaces that facilitate rotation and interface within attached tulip connectors 116.

The tulip connector 116 includes a tulip housing 118, a split-ring 120, a bushing 122 and a locking actuator 126. The tulip housing 118 is hollow with opposed slots 124 extending down a portion of opposing walls resulting in a tulip shaped housing. As shown in FIG. 10, the tulip housing 118 has a longitudinal bore therethrough defining a socket 127 with threaded interior walls at a proximal end of the tulip housing, and an inwardly extending flange 128 about a distal end of the tulip housing. The socket 127 is sized to receive the bulbous end 114 of the bent shaft 112, with the bulbous end rotatably engaged within the socket.

The split-ring 120 includes a material gap along its circumference that allows the split-ring to expand to a larger diameter for placement around the bulbous end 114 and to close and seat at the distal end against the flange 128. At its seated position against the flange 128, the split-ring 120 closes its expanded opening to a diameter less than the diameter of the bulbous head and keeps the extended range coupler 110 and tulip housing together with the split-ring holding the larger diameter bulbous head within the socket 127.

The bushing 122 includes a substantially concave inferior surface shaped complementary to the shape of the bulbous end 114, and a concave superior surface shaped to match the shape of the frame rod 108. The bushing 122 is placed into the socket 127 above/outside the bulbous end 114. The bushing shown in FIG. 10 can have a diameter larger than a diameter of the threaded interior walls of the socket surface to fit in the socket below the threaded surface and above the bulbous end 114. This fitting helps to hold the bushing 122 within the socket 126 below the threaded surface.

The frame rod 108 may be coupled to the tulip connector 116 by inserting the frame rod into the opposed slots 124 and onto the bushing 122. A locking actuator 126 is threadedly engaged with the socket threaded interior walls to loosen or tighten the bulbous end 114 to the frame rod. As such, the locking actuator 126 is operable to release and fixedly secure the frame rod 108 to the bent shaft 112 in response to mechanical loosening or tightening inputs. At the opposite end of the bent shaft 112, a second tulip connector 130 may likewise be coupled to a bone fastener 106, as will be described in greater detail below.

The tulip connectors 116, 130 when loosened allow for polyaxial movement between connected bone frame assembly components (e.g., frame rod 108, bent shaft 112, bone fastener 106) with three-dimensional full articulation. As such, the surgical bone frame assembly 100 may be adjustably connected and change shape, orientation, size and stiffness in response to mechanical loosening or tightening inputs between adjustable and locked configurations. In an unlocked or loose configuration, the connected bone frame assembly components may be adjusted to any preferred or predetermined position. Once a desired patient positioning is achieved, locking actuators 126 of the adjustable connectors may be engaged to secure the connections and create a rigid framework to maintain the patient position in the desired or predetermined position.

The bone fastener 106 is operable for securely attaching to the bone (e.g., bone segments, spinous process including posterior elements of the vertebrae, large bone, femur, iliac) of the patient. In accordance with an exemplary embodiment, the bone fastener 106 includes a clamp 132 that is fixed to an elongated bone fastener rod 134 having a longitudinal axis that extends substantially parallel to a longitudinal axis of the clamp. In some examples the elongated bone fastener rod may have a bend e.g. of about 5-60 degrees, including 10, 20, 30, 40 and 50 degrees to allow articulation and spacing from the clamp. The bone fastener rod 134 connects the bone fastener 106 to the second tulip connector 130 by inserting the bone fastener rod into the opposed slots 124 and onto the bushing 122 of the second tulip connector. The bone fastener rod 134 may be straight or bent to allow for greater connecting flexibility to the second tulip connector and the patient bone. A locking actuator 126 is threadedly engaged with the socket threaded interior walls to loosen or tighten the bulbous end 114 to the bone fastener rod. As such, the locking actuator 126 is operable to release and fixedly secure the bone fastener rod 134 to the bent shaft 112 in response to mechanical loosening or tightening inputs. With the adjustable tulip connectors 116, 130 connecting the extended range coupler 110 to the frame rod 108 and the bone fastener 106, respectively, the adjustable tulip connectors articulatively and fixedly connect the bone support frame to bone fasteners.

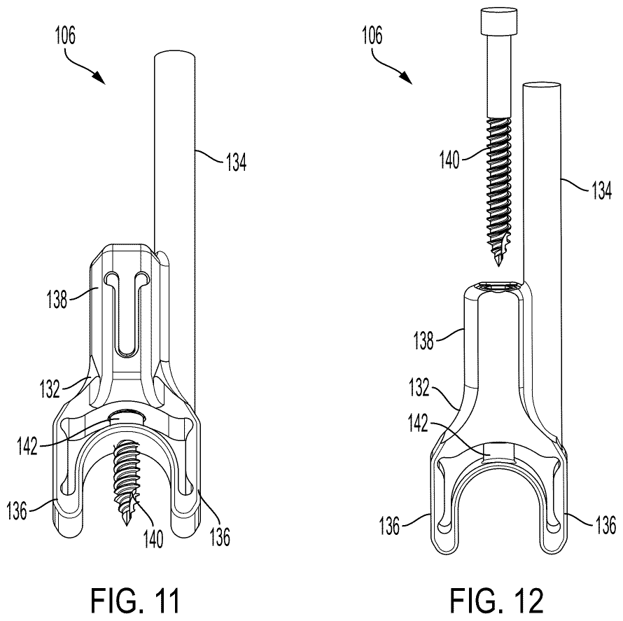

As shown in FIGS. 11 and 12, the bone fastener 106 has a distal end operable to attach to a bone or bone segments of the patient, and may hold the bone in a fixed position as desired during surgery. For example, the bone fastener 106 includes the clamp 132 that is generally U-shaped with opposing clamp arms 136 at its distal end to releasably engage and hold a bone. The clamp arms may be fixed arms or alternatively be configured to be adjustable arms e.g., pivotable or pivoting clamp arms. The clamp 132 includes an elongated tubular sleeve 138 for receiving a threaded screw 140 operable to allow a tip of the threaded screw to pass through and engage bone encompassed by the clamp arms. The threaded screw may also rotate and draw the clamp arms together to tighten and secure the bone or apart to loosen a grip, as readily understood by a skilled artisan. The elongated tubular sleeve defines a central aperture in fluid communication with an areas between the clamp arms 136.

While the bone fastener 106 in FIGS. 8, 9, 11 and 12 is shown as a U-shaped clamp, it is understood that the bone fastener is not limited to any particular shape or size, and may be oriented to accommodate variations in patient anatomy and positioning requirements. Exemplary clamps may include set screws, cam-actuated clamps, or other mechanical fastening systems that apply radial compression to the bone. The clamping force may be sufficient to prevent movement of the bone fastener under surgical loads while allowing for repositioning when the bone fastener may be loosened. The clamps may incorporate aspects such as multiple contact points or distributed clamping surfaces to provide uniform force distribution and prevent damage to the bone surface during clamping operations. The clamps may include a threaded screw for secure attachment to the patient, for example, by screwing into or abutting against the bone.

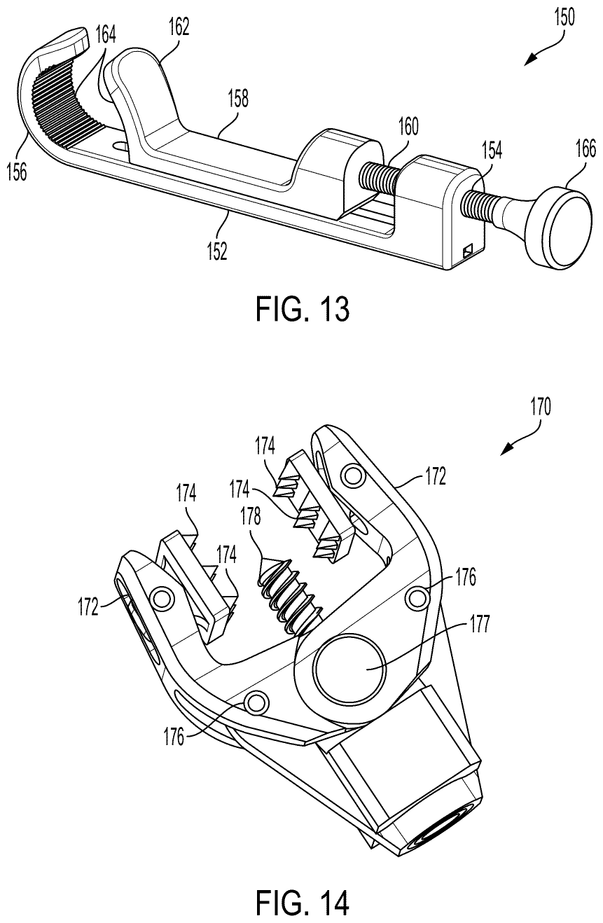

FIGS. 13-17 illustrate additional exemplary bone fasteners applicable to the subject disclosure that may be used to fixedly attach to a bone or bone segment of the patient. The bone fasteners are mechanical clamps operable for controlled gripping and stabilization of anatomical structures, such as bone and bone segments. FIG. 13 illustrates an exemplary C-clamp bone fastener 150 that may be used for stabilizing long bones. The C-clamp bone fastener 150 includes a base 152 having a curved jaw 156 about its distal end. An adjustable arm 158 is slidably attached to the base via a screw 160 operatively engaged with a proximal end of the base. The adjustable arm 158 includes a curved jaw 162 about its distal end. The adjustable arm 158 slides along the base 152 upon rotation of the screw 160 threadedly engaged in the threaded aperture 154 of the base and allows tightening and loosening of the clamp. The curved jaws 156, 162 include textured inner surfaces 164, e.g., ridges to facilitate gripping of bone. The screw 160 may include a knob 166 to help facilitate user rotation of the screw.

FIG. 14 illustrates an adjustable jaw clamp 170 for an exemplary bone fastener of the subject disclosure that may be used for stabilizing bone of a patient. The jaw clamp 170 includes two opposing arms or jaws 172. A board having teeth 174 is pivotably connected to the jaws and faces interiorly of the jaw clamp. The jaws 172 are adjustable and can rotate about pivot axes 176, 177 to open and close smoothly, enabling precise control during application. The teeth 174 are serrated to enhance grip and prevent slippage while operatively gripping a bone. The jaw clamp 170 also includes a central screw 178 located between the jaws that can engage and lock the bone with additional radial pressure.

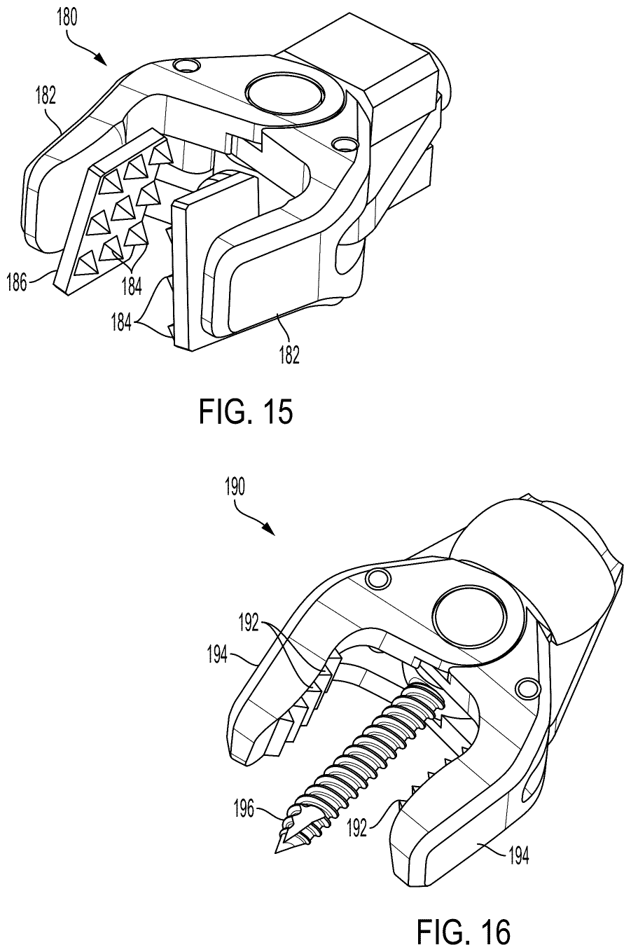

FIG. 15 depicts another exemplary embodiment of an adjustable jaw clamp 180 similar to the jaw clamp 170 illustrated in FIG. 14. The jaw clamp 180 is also operational for firm, non-slip engagement with anatomical structures such as bones. The jaw clamp includes opposing jaws 182 with serrated teeth 184 mounted to a support board 186 that permits three-dimensional articulation of the board and teeth for secure engagement with irregular surfaces. For example, the support board 186 may be coupled to the opposing jaws 182 via a ball and socket adjustable connection.

FIG. 16 depicts yet another exemplary embodiment of an adjustable jaw clamp 190 similar to the jaw clamp 170 illustrated in FIG. 14. The jaw clamp 190 includes serrated or ridged teeth 192 on its inner or medial surfaces of opposing jaws 194 for securely gripping bone, and a central screw 196 between the jaws manually adjustable to help lock the clamp in place.

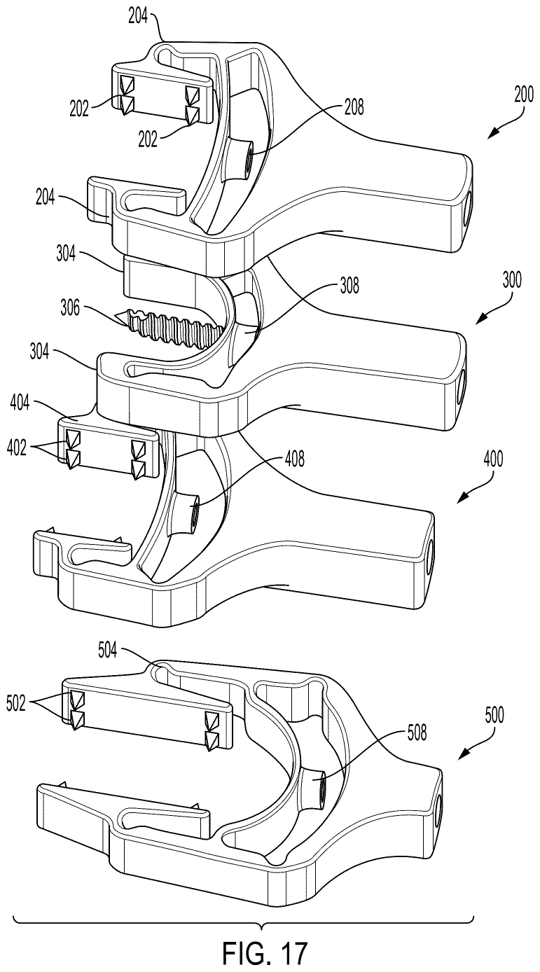

FIG. 17 illustrates a plurality of exemplary adjustable jaw clamps 200, 300, 400, 500 operational for firm, non-slip engagement with anatomical structures such as spinous process bones. Jaw clamps 200, 400 and 500 each include at least one of serrated teeth 202, 402, 502 on an inner surface of opposing jaws 204, 404, 504. The teeth of each jaw clamp can be configured on a plane or board 203, 403, 503 that is hingely connected to the jaw clap arms. The hinged connection can be e.g., a living hinge connection.

Each of the adjustable jaw clamps can include a central screw, such as central screw 306 (not shown for jaw clamps 200, 400, 500) extending between the jaws for engaging and locking a bone secured between the jaws. The screw 306 may also be used to adjust radial pressure to the bone by rotation within the inner wall 208 to loosen or lock the opposing jaws against the bone. In other words, the central screw can have a single function of operating the opposing jaws, or a dual function also as a threaded fixation into the bone. The dual functions can be adjusted such that one occurs before the other, or they are simultaneous.

Referring back to FIG. 1, the surgical bone frame assemblies 10, 100 may be used with the navigation system 1006 that may be configured for tracking a position of the patient and instruments used during surgery of the patient. In examples, the navigation system may include fiducial markers 80 operable for attachment to the patient via the surgical bone frame assembly and a navigation sensor 1006 configured to detect the position and orientation of the fiducial marker. The fiducial markers 80 may communicate with the navigation sensor 1006 and controller 1008 such that the location and orientation of the fiducial markers may be tracked to determine patient anatomical orientation and patient movement adjacent the bone frame assemblies 10, 100.

Fiducial markers are typically small objects, often a metal, reflective material, LED, RFID tag, etc., that may be attached (e.g., adhesive, molded, soldered, press fit, embedded, sprayed, deposition) to bone fasteners 20 that are attached to the patient. Fiducial markers may also be attached to the frame 12, frame fastener 14, and/or the bone support assembly 102.

While not being limited to a particular theory, the navigation system 1006 may include an optical tracking system, an electromagnetic tracking system and/or another tracking system configured to track the position and orientation of the fiducial marker attached to the bone frame assemblies 10, 100 attached to the patient. For an optical tracking system, the navigation system may include a camera (e.g., infrared (IR)-sensitive, Charge-Coupled Device (CCD), Complementary Metal-Oxide-Semiconductor (CMOS)) that captures the 2D positions of the fiducial marker with line-of-sight visibility. An optical fiducial marker 80 may be active and emit IR light (e.g., LEDs) and/or may be passive and reflect IR light from an external source. With a second camera offset from the first camera, the navigation system 1006 via controller 1008 may use triangulation to reconstruct the position and orientation of the fiducial marker for real-time tracking of the marker.

For an electromagnetic tracking system, the navigation system 1006 may include a transmitter that emits a low-frequency electromagnetic field in the tracking space around the patient, and the fiducial markers 80 may include a receiver (e.g., tiny coil embedded therein) that detects the magnetic field and communicates with the controller 1008 to process the signals and track the receivers without requiring line-of-sight visibility. It is understood that the navigation system 1006 is not limited to optical tracking systems or electromagnetic tracking systems, as other tracking systems may also be used within the scope of the examples. Such other tracking systems may include inertial tracking systems that may use combinations of accelerometers, gyroscopes and magnetometers; ultrasound-based tracking systems that may use ultrasound pulses and time-of-flight measurements; radio frequency (RF) and RFID tracking; and hybrid systems that combine multiple tracking modalities. A hybrid approach may allow the navigation system to maintain tracking continuity when one tracking system may be temporarily compromised by patient position, anatomical obstructions (e.g., line-of-sight) or surgical procedures.

The surgical bone frame assemblies 10, 100 may operate in different modes depending on surgical requirements and procedural phase. In some cases, the surgical bone frame assemblies may stabilize an otherwise unstable patient anatomical feature (e.g., spine, femur, pelvic region, torso, etc.). For example, the bone frame assemblies may be manually positioned to an initial or predetermined configuration and then set in place, that is, locked via locking the adjustable connectors to hold the bone fasteners in place, and then attached to a patient. The initial configuration may be a preferred, predetermined or ideal position for surgery or other intended purposes. The initial position may also be an unlocked configuration from which the adjustable connectors remain responsive to user input for dynamic patient positioning. In some cases, after initial positioning the adjustable connectors may be loosened to allow repositioning. Transition between locked (i.e., tightened) and unlocked (i.e., loosened) modes may be controlled through user adjustments to the adjustable connector locking actuators. The dynamic adjustment capability allows for real-time positioning and repositioning of the bone frame assemblies to acquire and maintain preferred and ideal patient positioning proactively prior to instrumentation and reactively throughout instrumentation as conditions and undesired patient movement occur.

During operation, the bone frame assemblies can initially function in a setup mode where surgical personnel manually attach the bone frame assembly to the patient. To help establish position references, any number of fiducial markers may be attached to the patient and bone frame assembly components at the anatomical region of interest. The navigation system 1006 may begin real-time tracking of the fiducial markers for patient position monitoring. Patient position data, acquired via continuous sensor measurements of fiducial marker location and orientation, is processed by the controller. Accordingly, patient position may be known as the bone frame assembly components are positioned to an ideal or predetermined orientation, locked in place to hold the patient in the ideal or predetermined orientation, unlocked to allow further patient movement, and moved to reposition the patient as needed. It is understood that even when a patient is locked in an ideal or predetermined position by the surgical bone frame assembly, the patient may still move, and the patient's previously stable position may become unstable due to the movement. For example, a patient's spine may move when the surgical bone frame assemblies are locked due to breathing, muscle relaxation under anesthesia, or external forces applied during surgery. In such cases, the controller 1008 in communication with the navigation system may determine if the position of the patient has moved from the predetermined position based on the tracked position of the fiducial marker, and adjust the position of the bone frame assembly to realign the patient to the predetermined position when it is determined that the patient has moved from the predetermined position. In other words, upon detection of undesired patient movement, the controller may calculate positioning adjustments, and coordinate movement of the surgical bone frame assembly or assemblies to reposition the patient to the ideal position, or to another desired position.

The frame segments or members of the surgical bone frame assembly may be configured as rigid structural elements that maintain their shape and dimensional relationships under the forces encountered during surgical procedures. These frame segments may be constructed from biocompatible materials such as stainless steel, titanium alloys, or other materials that provide the necessary strength and stiffness while maintaining compatibility with the surgical environment. The frame segments may be designed with specific cross-sectional geometries to provide optimal strength-to-weight ratios while allowing for sterilization and cleaning procedures required in surgical applications. The length and configuration of individual frame segments may be selected based on the specific anatomical region being supported and the spacing between attachment points on the patient's anatomy.

Adjustable connectors may enable the surgical bone frame assembly to accommodate variations in patient anatomy and positioning requirements while maintaining structural integrity throughout the surgical procedure. The adjustable connectors may allow for initial positioning and alignment of the surgical bone frame assemblies to match the patient's specific anatomical configuration, followed by locking or securing of the connectors to create a rigid structural framework at a predetermined orientation or position. The adjustable nature of the connectors accommodates differences in patient size, anatomical variations, and positioning requirements for different surgical procedures while providing the flexibility needed for initial bone frame assembly and setup.

Tulip connectors may provide benefits to frame assembly components that offer adjustability, structural performance, and ease of use. The tulip connectors may allow frame assembly components to be connected at various angles and positions, providing multi-directional adjustment capabilities while maintaining secure connection to the frame structure. It is understood that the adjustable connectors are not limited to any particular shape, size or configuration, and may be oriented to accommodate variations in patient anatomy and positioning requirements

It will be appreciated by those skilled in the art that changes could be made to the various aspects described in the above exemplary embodiments without departing from the broad inventive concept thereof. It is to be understood, therefore, that the subject application is not limited to the particular aspects disclosed, but it is intended to cover modifications within the spirit and scope of the subject application as defined by the appended claims

We claim:

1. A surgical bone frame assembly comprising:
a frame that includes a plurality of frame segments having adjustable connectors for operatively and articulatively connecting to each other;
a frame fastener for securing the frame to a patient; and
a bone support assembly connectable to the frame for fastening to a bone of a patient, the bone support assembly including:
a bone support frame for rigidly connecting to the frame, and
a bone fastener extending from the bone support frame for securely attaching to the bone of the patient.

2. The surgical bone frame assembly of claim 1, wherein the bone fastener includes a clamp.

3. The surgical bone frame assembly of claim 1, wherein the bone fastener includes a threaded screw.

4. The surgical bone frame assembly of claim 1, wherein the bone fastener includes a C-clamp.

5. The surgical bone frame assembly of claim 1, wherein the bone fastener includes a jaw clamp.

6. The surgical bone frame assembly of claim 1, wherein the bone fastener includes a pair of opposing pivotable clamp arms each having a distal end with a serrated inner surface.

7. The surgical bone frame assembly of claim 1, wherein the bone fastener is a screw or a pin.

8. The surgical bone frame assembly of claim 1, wherein the plurality of frame segments includes a first plurality of frame segments extending between a pair of the plurality of frame segments.

9. The surgical bone frame assembly of claim 1, wherein the bone support frame includes an elongated rod and an adjustable connector articulatively and fixedly connecting the elongated rod to the frame.

10. The surgical bone frame assembly of claim 1, wherein the bone support frame of the bone support assembly includes a lattice structure of a plurality of frame segments connected via adjustable connectors therebetween, the adjustable connectors articulatively and fixedly connecting the frame segments to the bone fastener.

11. The surgical bone frame assembly of claim 1, further comprising a fiducial marker operatively connected to the bone fastener, bone support assembly and/or frame.

12. A surgical bone frame assembly comprising:
a bone support assembly for securing an orientation of bone segments of a patient, the bone support assembly including:
a bone support frame;
a plurality of bone fasteners extending from the bone support frame for securely attaching to the bone segments of the patient; and
adjustable connectors articulatively and fixedly connecting the bone support frame to the plurality of bone fasteners.

13. The surgical bone frame assembly of claim 12, wherein the bone support frame includes a plurality of rigid frame segments, and the adjustable connectors operatively connect the plurality of rigid frame segments and the plurality of bone fasteners.

14. The surgical bone frame assembly of claim 12, wherein the bone support frame includes an elongated rod and an adjustable frame connector articulatively and fixedly connecting the elongated rod to an exterior frame.

15. The surgical bone frame assembly of claim 12, wherein the bone support frame of the bone support assembly includes a lattice structure of a plurality of frame segments connected via at least one adjustable support frame connector therebetween, the adjustable connectors articulatively and fixedly connecting the frame segments to the bone fastener.
